(19) United States
(12) Patent Application Publication
(10) Pub. No.: US 2006/0045893 A1
(43) Pub. Date: Mar. 2, 2006

(54) Long-wearing cosmetic compositions

(76) Inventors: Warren Hwa-lin Yu, Owings Mills, MD (US); Haiminot Bekele, Baltimore, MD (US); Donald Frank Rainey, Owings Mills, MD (US); Ronald Alan Sanford, York, PA (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/927,994

(22) Filed: Aug. 27, 2004

(51) Int. Cl. A61K 8/02

(52) U.S. Cl. 424/401

(57) ABSTRACT

A composition, a method for using said composition and a packaging system for said composition to significantly improve the appearance and feel of long wearing film-forming basecoats wherein said composition is a multi-layer cosmetic composition comprising: (a) a first composition, transfer-resistant, film-forming basecoat formulated to be applied to keratinous tissue to form a basecoat film thereon; and (b) a second, topcoat composition comprising a plasticizing agent wherein, the basecoat film, after application of said second, topcoat composition, has a percent L value change of the basecoat film of less than about 50% and a percent L value change of a transfer substrate greater than about 9% to about 30%, as determined by the rub test.

### FIELD OF THE INVENTION

This invention is for a composition, a method for using said composition and a packaging system for said composition to significantly improve the appearance and feel of long wearing film-forming basecoats.

### BACKGROUND OF THE INVENTION

Two-step lip color systems that provide better long wear performance than traditional lipsticks are in great demand. These systems use a pigmented basecoat to provide color and wear while the secondary topcoat provides gloss and lubricity. While these systems have significant improvement in wear, negatives still exist with respect to feel and appearance. For example, the product may feel dry and tight while also appearing matte. A matte appearance may be undesirable among users looking for a glossy, shiny look. Additionally, the product may wear off unevenly over time, resulting in an undesirable appearance. In order to improve feel, some consumers may use lip balm or similar products to help alleviate the dryness and tightness. These products may help improve the feel and aesthetics of the basecoat film but are not optimized to enhance feel and appearance without impacting wear.

In order to alleviate the dryness and tightness of the long wearing film, a plasticizer can be incorporated into the composition. The plasticizer creates a softer, more flexible film that feels more comfortable. While the use of plasticizers is common in many industries including personal care products, most plasticizers are incorporated within a polymer to form a product. Though this approach improves flexibility and feel, it compromises the adhesion to the substrate (keratinous surfaces). For example, U.S. Pat. No. 6,027,739, assigned to Lip-Ink International, published Jan. 21, 2003, discloses the use of castor oil as a potential plasticizer for an alcohol soluble and water insoluble resin. The resin is used in a smear resistant lip color to provide long wear attributes. The plasticizer is incorporated together with the resin during the making process, resulting in one vehicle containing both plasticizer and resin. U.S. Pat. No. 5,989,570, assigned to L'Oreal, published Nov. 23, 1999, discloses a cosmetic composition containing a plasticizing oligomer and a film-forming polymer and uses thereof. The plasticizers in accordance with the invention are incorporated into the composition as one vehicle. U.S. Pat. No. 4,795,631, assigned to Chesebrough-Pond's, Inc., discloses a water based lip color comprising an alkali soluble film forming agent that does not smear or bleed. A water insoluble plasticizer is incorporated into the composition during the making process and is used to create a more flexible film. Lastly, U.S. patent application Ser. No. 10/066,055, Scancarella et al., assigned to Revlon, Inc., discloses a method for improving the aesthetics of a pigmented transfer resistant film on the lips wherein said method comprises coating the transfer resistant film with a non-reactive wetting agent composition. The wetting agent composition wets the transfer resistant film, which in turn provides hydration and comfort to the composition of the lips. Although the non-reactive wetting agent composition helps improve the feel and appearance, these attributes are inferior when compared to a conventional lip color. On the other hand, a conventional lip color does not contain the necessary amount of polymer to exhibit transfer-resistance properties. Therefore, a need exists for compositions wherein there is a reactive topcoat that may significantly improve the feel and appearance while maintaining a sufficient degree of transfer resistance.

Plasticizers incorporated in the bulk product, such as those described in the references above, soften or plasticize the polymer but compromise the adhesion of the polymer to the substrate. Consequently, the present invention has found a two layer cosmetic composition and system wherein the plasticizer is kept separate from the polymer to be plasticized until after polymer film formation. As a result, the polymer is able to form a transfer resistant film with optimal adhesion to the substrate (i.e., keratinous surfaces) without any plasticizer. Upon application of the plasticizer over the film-forming polymer, the surface of the polymer film softens while the adhesion between substrate and polymer remains intact. Ultimately, the polymer is plasticized without significantly impacting the transfer resistance properties of the film forming basecoat.

### SUMMARY OF THE INVENTION

The present invention relates to a multi-layer cosmetic composition comprising:
a. a transfer-resistant, film-forming basecoat composition formulated to be applied to keratinous tissue to form a basecoat film thereon; and
b. a topcoat composition comprising a plasticizing agent wherein the basecoat film, after application of said topcoat composition thereon, has a percent L value change of the basecoat film of less than about 50% and a percent L value change of a transfer substrate greater than about 9% to about 30%, as determined by the rub test.

The present invention also relates to a method for improving the feel and aesthetics of transfer-resistant, film-forming basecoats wherein said method comprises the steps of:
a. applying a transfer-resistant, film-forming basecoat composition onto the surface of keratinous tissue to form a basecoat film;
b. allowing said basecoat to dry; and
c. applying over said basecoat a topcoat composition comprising a plasticizing agent wherein the basecoat film, after application of said topcoat composition thereon, has a percent L value change of the basecoat film of less than about 50% and a percent L value change of a transfer substrate greater than about 9% to about 30%, as determined by the rub test.

2. The present invention also comprises a system for improving the feel and aesthetics of transfer-resistant, film-forming basecoats wherein said system comprises cosmetic compositions comprising:
a. a transfer-resistant, film-forming basecoat composition formulated to be applied to keratinous tissue to form a basecoat film thereon; and
b. a topcoat composition comprising a plasticizing agent wherein the basecoat film, after application of said topcoat composition thereon, has a percent L value change of the basecoat film of less than about 50% and a percent L value change of a transfer substrate greater than about 9% to about 30%, as determined by the rub test; and
c. instructions indicating that the composition of (a) should be used with the composition of (b).

### DETAILED DESCRIPTION OF THE INVENTION

While the specification concludes with the claims particularly pointing and distinctly claiming the invention, it is believed that the present invention will be better understood from the following description.

As used herein, “comprising” means that other steps and ingredients can be added. This term encompasses the terms “consisting of” and “consisting essentially of”. The phrase “consisting essentially of” means that the composition may include additional ingredients, but only if the additional ingredients do not materially alter the basic and novel characteristics of the claimed compositions or methods.

All percentages, parts and ratios are based upon the total weight of the topical compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

As used herein, the term “transfer-resistant, film-forming basecoat” means a cosmetic product that forms a film upon application of the cosmetic product and is substantially transfer-resistant wherein the cosmetic product has increased wear. The cosmetic product can be applied to keratinous tissue, and may be in the form of a solid, liquid, powder, solid emulsion, conventional bullet, gel, cream, lip color pen, and the like.

The term “plasticizer” refers to a material applied to a given film-forming polymer to improve its flexibility or to soften the film. These are materials which, when applied to a given film forming-polymer, soften the polymer in either or both of the following means i) lowering the glass transition temperature, Tg, of the polymer system ii) lowering the complex modulus of the polymer system.

The term “lip product” means a cosmetic that can be applied to the lips and may be in the form of a liquid, conventional bullet, gel, cream, lip color pen, and the like.

The term “conventional lip color” means a cosmetic lip product that imparts color but readily transfers color to other objects upon contact.

The term “keratinous tissue,” as used herein, refers to keratin-containing layers disposed as the outermost protective covering of mammals which includes, but is not limited to, skin, lips, hair, toenails, fingernails, cuticles, hooves, etc.

The term “Complex modulus, G*”, is a measurement of flexibility. As the body or substance becomes more flexible, it will have less resistance to an applied strain.

As used herein, “drawdown” refers to a basecoat film of uniform thickness on a substrate.

The term “L value”, also known as “L* value”, is a measure of the lightness of an object based on the color model developed by the CIE (Commission Internationale de l'Eclairage) committee. The L value ranges on a scale of 100 to 0, 100 being the lightest and 0 the darkest. The compositions of the present invention necessarily comprise the following ingredients.

The present invention comprises a composition, method and packaging system of improving the aesthetics and feel of long wear lip color products by wetting the basecoat film of said lip color product with a topcoat that contains a plasticizer. It has been discovered that the application of certain materials or plasticizers, wherein said materials or plasticizers have a slight compatibility or solubility with the basecoat film, over a transfer-resistant film, improve the aesthetics and feel compared with an incompatible or nonplasticizing material. The feel advantage comes without significantly impacting the long wear performance of the film forming basecoat.

The present invention uses a method whereby first, a basecoat is applied to keratinous tissue and allowed to dry in order to adhere to the substrate. Secondly, a plasticizing topcoat is applied which begins to plasticize the basecoat polymer upon application. The plasticized polymer results in an interphase layer between the basecoat and topcoat, wherein said interphase layer comprises both polymer and plasticizer. The interphase layer is more flexible and softer due to the plasticization of the polymer within the basecoat, resulting in improved feel. The polymer at the substrate/basecoat interface remains unplasticized, thus retaining the transfer-resistant and adhesive properties to the lip or skin surface. The topcoat layer remains at the surface providing lubricity and shine. The plasticizing topcoat may also contain pigments and pearls, which help retain color and improve shine of the lip surface. The overall result is improved feel and appearance for long wearing lip color.

Basecoat

The present invention comprises a basecoat composition containing one or more polymers that provide adhesive and transfer resistance properties to the surface of keratinous tissue. Such compositions generally contain one or more polymers or resin in combination with a volatile carrier. The polymer can be any polymer or resin that provides some transfer resistance properties for the basecoat. Suitable polymers, copolymers, or resins for the present invention include, but are not limited to silicone resins, silicone polymers, polysaccharides, polyurethanes, acrylates, polyesters, alkyds, fluoro polymers, and mixtures thereof. Preferably, the resin is an organosiloxane resin and the polymer is a fluid diorganosiloxane polymer.

Organosiloxane Resin

The compositions of the present invention may comprise an organosiloxane resin. The resin may comprise combinations of R₃SiO_{1/2} “M” units, R₂SiO “D” units, RSiO_{3/2} “T” units, SiO₂ “Q” units in ratios to each other that satisfy the relationship RₙSiO_{(4-n)/2} where n is a value between 1.0 and 1.50 and R is a methyl group. Up to 5% of silanol or alkoxy functionality may also be present in the resin structure as a result of processing. The organosiloxane resins have a number molecular weight average range of from about 1,000 g/mole to about 10,000 g/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrafunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is about 0.6 to about 2.0, more preferably about 0.6 to about 0.9, most preferably about 0.7. Examples of organosiloxane resins commercially available are Wacker 803 and 804 available from Wacker Silicones Corporation of Adrian Mich., and G.E. SR1000 from the General Electric Company.

The organosiloxane resins are used in the present invention at levels from at least about 10%, preferably from at least about 55%, more preferably from at least about 60% and no more than about 95%, preferably no more than about 80%, and more no more than about 70% of the total amount of organosiloxane resin, diorganopolysiloxane polymers and modified silicones.

Diorganopolysiloxane Polymer

The present invention may also employ a diorganopolysiloxane polymer that is combined with the organosiloxane resin disclosed above. Applicants have found that suitable polymers exhibit a viscosity of at least about 1 cSt at 25° C. The diorganopolysiloxane polymers of the present invention comprise repeating units, wherein said units correspond to the formula (R₂SiO), where R is a monovalent hydrocarbon radical containing from 1 to 6 carbon atoms, preferably selected from the group consisting of methyl, ethyl, propyl, isopropyl, butyl, isobutyl, t-butyl, amyl, hexyl, vinyl, allyl, cyclohexyl, phenyl, fluoroalkyl and mixtures thereof. The diorganopoylsiloxanes employed in the present invention may contain one or more of these hydrocarbon radicals as substituents on the siloxane polymer backbone. The diorganopolysiloxanes may be terminated by triorganosilyl groups of the formula (R′₃ Si) where R′ is a radical selected from the group consisting of monovalent hydrocarbons containing from 1-6 carbon atoms, hydroxyl groups, alkoxyl groups and mixtures thereof. When a diorganopolysiloxane polymer is present, it is essential that it be compatible in the mixture with the organosiloxane resin material and the volatile carrier. The term “compatible” refers to the formation of a homogeneous blend when the diorganopolysiloxane, organosiloxane resin and volatile carrier are mixed together in ratios required for a specific formulation. A particularly preferred diorganopolysiloxane polymer is poly(dimethylsiloxane), herein referred to as PDMS. Suitable diorganopolysiloxane polymers have the following structural formula wherein n is equal to from about 1500 to about 4500: 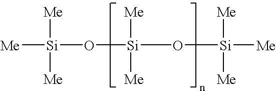
Topcoat

The topcoat composition of the present invention contains one or more plasticizers that when applied to the basecoat plasticize the polymer and creates a softer, more flexible film without impacting adhesion to the substrate. The degree of plasticization and transfer resistance can be measured by the rub test, described herein. The rub test has been developed to clearly demonstrate the distinctiveness of the present invention in terms of the physical characteristics of the films formed from such compositions.

Typically, the lower the viscosity and/or the complex modulus, the more likely the plasticizer will have a higher interaction with the polymer, thus lowering the Tg of the polymer. If the viscosity is low enough, the plasticizer will become more like a solvent, disrupting the film completely. If the amount, however, is too low, the plasticizer may not soften the film at the topcoat/basecoat interface. Thus, topcoat compositions of the present invention comprise plasticizers from at least about 1%, preferably at least about 15%, more preferably at least about 30% and no more than about 99%, preferably no more than about 75%, more preferably no more than about 60%, by weight of the topcoat composition. The plasticizers are found to interact with the basecoat polymers at a viscosity from at least about 1 cSt.

Rub Test Method:

The primary benefit of long wearing lip color products is their ability to withstand various insults, such as abrasion, eating, drinking, kissing, etc. and still remain on the surface of the lip. To test the transfer resistance or wear properties of a lip color product, the present invention provides a Rub Test Method. This method tests the ability of the lip color product to resist color transfer on to a substrate as a result of abrasion. Using a color computer, the amount of product transferred is measured by observing the color change of the lip color prior to and after abrasion. The method is recorded by a reading of the L value of the basecoat film and the transfer substrate, which relates to the lightness of the basecoat color.

An increase in the L value from initial to final reading of the drawdown corresponds to a lightening of the film, indicating the film has been removed or “worn” to some degree. For example a composition that has an initial L reading of 50.0 of the drawdown and ends with an L value of 75.0 of the drawdown exhibits a percent L value change of 50%. Compositions of the present invention have a percent increase in L value less than about 50%, preferably less than about 35%, more preferably less than about 20%.

A decrease in the L value from initial to final reading of the transfer substrate corresponds to a darkening of the transfer substrate, indicating that some of the basecoat film has transferred to the substrate. The upper layers of the basecoat film are being plasticized in the present invention, therefore, some amount of transfer will occur during abrasion. The higher the degree of plasticization, the more the basecoat film will be transferred. The lower the degree of plasticization of a long wearing basecoat film, the less basecoat film will be transferred. Compositions of the present invention have a percent decrease in L value of greater than 9%, more preferably greater than 12%, even more preferably greater than 15%, but no more than 30%.

Equipment:

Datacolor Microflash computer 200d or equivalent
2.5 inch circular punch (Arch Punch-Macarchco Company)
Leneta Card—Form 2A Opacity Chart
2 kg stainless steel cylindrical weight with 2.5 inch circular diameter
Rubber hammer
Kimberly Clark WypAll L40 All Purpose Wipers
Double sided adhesive tape
0.001″ drawdown bar (Bird applicator−1 mil (0.001″)×3″ width)
Rubber spatula
   Sample Preparation:

(1) Prepare 3 drawdowns on a Leneta card for each prototype to be tested (see table below). Use a 0.001″ drawdown bar. Solid products should be sheared until sufficiently liquified for drawdown purposes.

(2) Dry drawdowns in a 50° C. oven for two hours.

(3) After films are dry, remove from oven.

(4) For the plasticizing topcoat prototype only, apply plasticizing topcoat on top of 3 of the films using a spatula. Care should be taken so as not to scratch the basecoat films during topcoat application. Apply the topcoat evenly, such that it completely covers the entire basecoat area to be measured.

(5) After applying topcoat, allow all drawdowns to set for 2 hours at ambient temperatures (approximately 24° C.-27° C.).

(6) After topcoat has set for 2 hours, use high pressure air from a hose or tube to blow excess product off film. Continue to blot any excess away by using approximately 2-3 kg of force over a paper towel over the film. Be sure to remove as much excess as possible. There should be no basecoat film removed, only topcoat. Excess topcoat allowed to remain on the basecoat film surface could adversely affect measurements

Transfer substrate (disk) preparation:

(1) Use the circular punch and a rubber hammer to make 2.5 in. disks out of the Wypall wipers. Make one disk for each drawdown. Each disk should be one layer thick.

2 kg Weight Preparation:

(1) Prepare the 2 kg weight for use, by applying double coated tape across the entire surface of the bottom of the weight. Ensure that tape is applied, such that strips do not overlap. Avoid air pockets between weight and tape.

(2) Trim excess tape from around the perimeter of the base using a razor blade.

(3) Apply a transfer disk substrate to the taped portion of the 2 kg weight, such that it covers the entire taped portion of the base without wrinkles in the disk.

Measurement:

(1) Use Microflash computer to measure L value of transfer substrate (while adhered to 2 kg weight) and film (using white portion of card). Record values in table. Transfer substrates used in this test should have an initial L value of at least 85. Basecoat films used in this test should have an initial L value ranging from 40 to 60.

(2) Place 2 kg weight w/transfer substrate on drawdown and turn 720° (2 full revolutions). Weight should be centered over film where Microflash measured.

(3) Carefully remove the 2 kg weight, perpendicular to the sample drawdown, and place on table with transfer substrate disk side up.

(4) Measure L value of drawdown (white portion) and transfer substrate (while adhered to 2 kg weight). Reading should be in center of worn test area. Record values.

(5) Calculate percent change L values for both basecoat film and transfer substrate by using the following formula:
% L Change=(Final value−Initial Value)/Initial Value×100%
A negative percent L value change indicates darkening of the object and a positive percent L value change indicates a lightening of the object.

(6) Peel away the transfer disk substrate, and replace with a fresh disk

(7) Repeat steps 1-6 for second and third drawdown and average all three percent L values changes together for transfer substrate and basecoat film. Standard deviation for transfer disk L values should be less than 3%. Standard deviation for basecoat film L values should be less than 10%, preferably, less than 5%.

Topcoat/Plasticizing Agents

Oils

Compositions of the present invention comprise a second, topcoat composition that is applied atop a first, basecoat composition. The topcoat composition necessarily comprises a plasticizing agent, which may be an oil in a form ranging from solid to liquids. Preferably, liquid oils are used and have a viscosity greater than about 1 cSt, more preferably greater than about 10 cSt, even more preferably greater than about 50 cSt, even more preferably greater than about 100 cst. Regardless of the form, compositions of the present invention contain at least one oil which acts as a plasticizer for the film forming basecoat. The oils used in the present invention are selected from the group consisting of hydrocarbons, esters, silicones, functionalized silicones, fluid synthetic polymers, waxes, solid oils and mixtures thereof. Any materials within these classes may be used so long as it satisfies the criteria of not significantly disrupting the film of the film forming basecoat once applied, such that the film forming basecoat has some transfer resistance.

Hydrocarbons

Hydrocarbons useful in the present invention include, but are not limited to, straight chain hydrocarbons, isoparaffins, mineral spirits, polybutene, hydrogenated polyisobutene, hydrogenated polydecene, polydecene, squalene, petrolatum and mixtures thereof.

Glycols

Glycols useful in the present invention include, but are not limited to, butylene glycol, propylene glycol, glycerin, and mixtures thereof.

Esters

The composition of the present invention may comprise esters having the chemical structure RCO—OR′ where R and R′ are independent groups consisting of alkyl, alkyene, or alkoxy chains. Esters useful in the present invention include, but are not limited to, isopropyl myristate, isopropyl linoleate and mixtures thereof.

Silicones

Silicones useful in the present invention include, but are not limited to, diorganosiloxanes, dimethiconols, cyclomethicones, alkyl silicones, phenyl trimethicone and mixtures thereof. Further examples of silicones useful in the present invention can be found in the CTFA Handbook, Chemical Classes, under Siloxanes and Silanes.

Compositions of the present invention may also comprise modified silicones. Particularly, the modified silicones are selected from the group consisting of aminosilicones, carboxy modified silicones, epoxy modified silicones and mixtures thereof. Modified silicones useful in the present invention have the following general structure wherein R can be selected from the group consisting of amino, epoxy or carboxy groups: 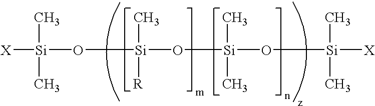
Aminosilicone R═—R′NH₂ or R═R′NHR″NH₂
Epoxy modified 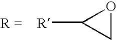
Carboxy modified R═R′COOH
R′ and R″=ethylene, propylene, isobutylene, butylene, isopropylene, alkylene, cycloalkylene alkylene ether
X═CH₃, OCH₃, R, or a combination thereof
   wherein m is 1-5, preferably 1-3, n is 10-100, and z is 8-450.
   Additionally, compositions of the present invention may comprise silicone modified fluorinated polymers used alone or in combination with the modified silicones disclosed above.

Fluid Synthetic Polymers

Fluid synthetic polymers useful in the present invention include, but are not limited to, polydecene, hydrogenated polyisobutene, polyisobutene, polybutene and mixtures thereof.

Waxes

The waxes useful in the present invention are herein defined as organic mixtures or compounds of high molecular weight, solid at 23° C. Generally waxes are similar in composition to fats and oils except that they contain no glycerides. Waxes include high molecular weight hydrocarbons, fatty acids, fatty acid esters, fatty alcohols and mixtures thereof. Waxes useful in the present invention include wax generally known for use in the cosmetic arts. Such waxes include those disclosed in U.S. Pat. No. 5,599,547 Bartholomey et al., issued Feb. 4, 1997.

Other waxes useful in the present invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof wherein the waxes have a melting point greater than about 30° C. Waxes useful in the present invention are selected from the group consisting of synthetic waxes, ozokerite, jojoba esters, “Unilins”, available from Petrolite Corporation, fatty alcohols from C22 to C50 and mixtures thereof. Synthetic waxes include those disclosed in Warth, Chemistry and Technology of Waxes, Part 2, 1956, Reinhold Publishing; herein incorporated by reference. The waxes most useful herein have melting points from about 30° C. to about 115° C. and are selected from the C 8 to C 50 hydrocarbon waxes. Such waxes include long chained polymers of ethylene oxide combined with a dihydric alcohol, namely polyoxyethylene glycol. Such waxes include carbowax available from Carbide and Carbon Chemicals company. Other synthetic waxes include long-chained polymers of ethylene with OH or other stop length grouping at end of chain. Such waxes include the Fischer-Tropsch waxes as disclosed in the text disclosed above at pages 465-469 and include Rosswax, available from Ross company and PT-0602 available from Astor Wax Company. Additional synthetic waxes include the class of alkylated polyvinyl pyrrolidones or PVP, including tricontanyl PVP (available as Gannex WP-660 from ISP Company) and PVPIEicosene Copolymer (available as from ISP Company).

Specific waxes useful in the present invention are selected from the group consisting of beeswax, lanolin wax, shellac wax (animal waxes); carnauba, candelilla, bayberry (vegetable waxes); ozokerite, ceresin, (mineral waxes); paraffin, microcrystalline waxes (petroleum waxes); polyethylene, (ethylenic polymers); polyethylene homopolymers (Fischer-Tropsch waxes); C 24-45 alkyl methicones (silicone waxes); and mixtures thereof. Most preferred are beeswax, lanolin wax, carnauba, candelilla, ozokerite, ceresin, paraffins, microcrystalline waxes, polyethylene, C 24-45 alkyl methicones, and mixtures thereof.

Solid Oils

Solid oils useful herein are those which have a melting point of above about 30° C. to about 250° C., preferably from about 37° C. to about 100° C., more preferably from about 37° C. to about 80° C. As used herein the term “solid oils” refers to any oil or oil-like materials which are solids or semi-solids at temperatures of from about 200° C. to about 25° C., and have a solubility in water of generally less than about 1% by weight at 25° C. Examples of suitable solid oils include, but are not limited to, petrolatum, highly branched hydrocarbons, fatty alcohols, fatty acid esters, vegetable oils, hydrogenated vegetable oils, polypropylene glycols, alpha-hydroxy fatty acids, fatty acids having from about 10 to about 40 carbon atoms, alkyl amides of di and/or tri-basic carboxylic acids, n-acyl amino acid derivatives, and mixtures thereof. Solid oils useful in the cosmetic composition of the present invention are further described in U.S. Pat. No. 4,919,934, to Deckner et al., issued Apr. 24 1990.

Suitable highly branched hydrocarbons for use herein include hydrocarbon compounds having from about 17 to about 40 carbon atoms. Nonlimiting examples of these hydrocarbon compounds include squalane, cholesterol, lanolin, docosane (i.e. a C₂₋₂ hydrocarbon), and isoparaffins.

Volatile Carrier

In the present invention, the combination of the organosiloxane resin and diorganosiloxane polymer is to be easily transferred to the lip surface using a package/applicator. To achieve delivery, it is necessary that this combination above be incorporated into a carrier, preferably a volatile carrier which quickly volatilizes from the surface of the lips leaving the above-discussed thin-durable film. The volatile carrier must solubilize the organosiloxane resin and the diorganosiloxane polymer.

The volatile carrier comprises from at least about 10%, more preferably from at least about 15%, even more preferably at least about 20% of the overall composition and no more than about 90%, preferably no more than about 80%, even more preferably no more than about 70% of the overall composition.

Hydrocarbon oils useful in the present invention include those having boiling points in the range of 60-260° C., more preferably hydrocarbon oils having from about C₅ to about C₂₀ chain lengths, most preferably C₇ to C₁₆ isoparaffins. Of these isoparriffins most preferred are selected from the group consisting of isododecane, isohexadecane, isoeocosane, 2,2,4-trimethylpentane, 2,3-dimethylhexane and mixtures thereof. Most preferred is isododecane, which is also known as 2,2,4,6,6-pentamethylheptane. Isododecane that is suitable for inclusion in the compositions of the present invention is available from a number of sources, e.g., from Presperse, Inc. as Permethyl 99A.

Preferred volatile silicone fluids include cyclomethicones having 4, 5, and 6 member ring structures corresponding to the formula: 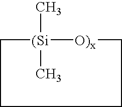
where X is from about 3 to about 6. Said volatile silicones include 244 Fluid, 344 Fluid and 245 Fluid, and 345 Fluid all from Dow Corning Corporation.
Optional Ingredients

There are a great number of other ingredients approved for use in the cosmetic art that may be used in compositions of the present invention. Such ingredients are those approved for use in cosmetics and can be found listed in reference books such as the CTFA Cosmetic Ingredient Dictionary & Handbook, Tenth Edition, The Cosmetic, Toiletries, and Fragrance Association, Inc. 2004. Said materials may be used so long as it satisfies the criteria of not significantly disrupting the film of the basecoat once applied, such that the basecoat has some transfer resistance. Said ingredients include particulates, fragrances, flavor oils, skin care ingredients such as sunscreen, emulsifiers and the like. Hypoallergenic compositions can be made into the present invention where said compositions do not contain fragrances, flavor oils, lanolin, sunscreens, particularly PABA, or other sensitizers and irritants. Oils are an important component of the present invention. In addition to said oils, other materials may be included to provide the product form desired by the consumer. Such forms include liquids, pastes, and solids. In the case of a solid form, the composition of the present invention comprises materials in a sufficient amount so as to form a stable stick. These materials are herein referred to as solid formers. Said solid formers are preferably used at levels from about 0.5% to about 35.0% more preferably from about 7.0% to about 25.0%, and most preferably from about 8% to about 20.0% of the composition. Said solid formers are selected from the group consisting of solid polyol fatty acid polyesters, waxes, solid oils and mixtures thereof.

Particulates

The compositions of the present invention may comprise one or more particulate materials. Nonlimiting examples of particulate materials useful in the present invention include particles, pigments, and cross-linked silicone elastomers.

Particulate materials useful herein include colored and uncolored pigments, interference pigments, inorganic powders, organic powders, composite powders, optical brightener particles, and combinations thereof. These particulates can be platelet shaped, spherical, elongated or needle-shaped, or irregularly shaped, surface coated or uncoated, porous or non-porous, charged or uncharged, and can be added to the current compositions as a powder or as a pre-dispersion. These particulate materials may provide a wide range of functions, including but not limited to modifying skin feel, masking the appearance of certain skin characteristics such as fine lines, wrinkles, and pores, improving application properties of the composition, masking the color of other components of the composition, and reducing migration of liquid materials on the skin. If used, particulate materials are present in the composition in levels of from about 0.01% to about 60%, more preferably from about 0.05% to about 50%, still more preferably from about 0.1% to about 30%, by weight of the composition. There are no specific limitations as to the pigment, colorant or filler powders used in the composition.

Particulate materials useful herein include but are not limited to bismuth oxychloride, sericite, mica, mica treated with barium sulfate or other materials, zeolite, kaolin, silica, boron nitride, lauroyl lysine, nylon, polyethylene, talc, styrene, polypropylene, polystyrene, ethylene/acrylic acid copolymer, sericite, aluminum oxide, silicone resin, barium sulfate, calcium carbonate, cellulose acetate, PTFE, polymethyl methacrylate, starch, modified starches such as aluminum starch octenyl succinate, silk, glass, fibers, ground seeds, pumice, and mixtures thereof. Especially preferred are spherical powders with an average primary particle size from about 0.1 to about 75 microns, preferably from about 0.2 to about 30 microns.

Suitable organopolysiloxane gel compositions are dimethicone/vinyl dimethicone crosspolymers swollen in an appropriate solvent. Such dimethicone/vinyl dimethicone crosspolymers are supplied by a variety of suppliers including Dow Corning (DC 9040™ and DC 9041™), General Electric (SFE 839™), Shin Etsu (KSG-15™, KSG-16™, KSG-18™ [dimethicone/phenyl vinyl dimethicone crosspolymer]) and lauryl dimethicone/vinyl dimethicone crosspolymers supplied by Shin Etsu (e.g., KSG-31™, KSG-32™, KSG-41™, KSG-42™, KSG-43™, and KSG-44™).

Alternatively, organopolysiloxane elastomer powders can be used, suitable examples include vinyl dimethicone/methicone silesquioxane crosspolymers like Shin-Etsu's KSP-100™, KSP-101™, KSP-102™, KSP-103™, KSP-104™, KSP-105™, hybrid silicone powders that contain a fluoroalkyl group like Shin-Etsu's KSP-200™, and hybrid silicone powders that contain a phenyl group such as Shin-Etsu's KSP-300™; and Dow Corning's DC 9506™.

Also useful herein are interference pigments. Interference pigments, for purposes of the present invention are defined as thin plate-like layered particles having two or more layers of controlled thickness with different refractive indices that yield a characteristic reflected color from the interference of typically two, but occasionally more, light reflections, from different layers of the plate-like particle. Examples of interference pigments are micas layered with about 50-300 nm films of TiO2, Fe2O3, silica, tin oxide, and/or Cr2O3. Such pigments are often pearlescent. Pearl pigments reflect, refract and transmit light because of the transparency of pigment particles and the large difference in the refractive index of mica platelets and, for example, the titanium dioxide coating. Useful interference pigments are available commercially from a wide variety of suppliers, for example, Rona (Timiron™ and Dichrona™), Eckart (e.g. Prestige and Prestige Silk lines). Especially preferred are interference pigments with smaller particle sizes, with an average diameter of individual particles less than about 75 microns in the longest direction, preferably with an average diameter less than about 50 microns.

Other pigments useful in the present invention provide color primarily through selective absorption of specific wavelengths of visible light, and include inorganic pigments, organic pigments and combinations thereof. Examples of useful inorganic pigments include iron oxides, ferric ammonium ferrocyanide, manganese violet, ultramarine blue, and Chrome oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO2, ZnO, or ZrO2, are commercially available from a number of sources. One example of a suitable particulate material contains the material available from U.S. Cosmetics (TRONOX TiO2 series, SAT-T CR837, a rutile TiO2). Particularly preferred are charged dispersions of titanium dioxide, as are disclosed in U.S. Pat. No. 5,997,887. The pigments/powders useful herein can be surface treated to provide added stability of color and/or for ease of formulation. Non-limiting examples of suitable coating materials include silicones, lecithin, amino acids, metal soaps, polyethylene and collagen. These surface treatments may be hydrophobic or hydrophilic, with hydrophobically treatments being preferred. Particularly useful hydrophobic pigment treatments include polysiloxane treatments such as those disclosed in U.S. Pat. No. 5,143,722.

Preferred lakes of the present invention are Red 3 Aluminum Lake, Red 21 Aluminum Lake, Red 27 Aluminum Lake, Red 28 Aluminum Lake, Red 33 Aluminum Lake, Yellow 5 Aluminum Lake, Yellow 6 Aluminum Lake, Yellow 10 Aluminum Lake, Orange 5 Aluminum Lake and Blue 1 Aluminum Lake, Red 6 Barium Lake, Red 7 Calcium Lake.

Other colors and pigments can also be included in the lip compositions, such as pearls, titanium oxides, Red 6, Red 21, Blue 1, Green 5, Orange 5 dyes, chalk, talc, iron oxides and titanated micas.

Emulsifiers

Emulsifiers may be used as coupling agents which have an affinity for the hydrophilic and hydrophobic phases of lip compositions of this invention. Emulsifiers are also useful for incorporating polar fluids such as water, propylene glycol, glycerine or mixtures thereof. Such emulsifiers include those routinely used in cosmetics and are found in the CTFA. Polar fluids such as water, glycerine, propylene glycol and mixtures thereof may also be incorporated without an emulsifier when amphiphilic materials such as polyol fatty acid polyesters are used in the composition.

Skin Care Active Ingredients

Skin care active ingredients in both water soluble and water insoluble forms can be added to the lip composition. Said ingredients include, but are not limited to, fat soluble vitamins, sunscreens and pharmaceutically active ingredients. These skin care active ingredients include, but are not limited to, glycerine, zinc oxide; chamomile oil; ginko biloba extract; pyroglutamic acid, salts or esters; sodium hyaluronate; 2-hydroxyoctanoic acid; sulfur; salicylic acid; carboxymethyl cysteine, and mixtures thereof

### EXAMPLES

The following examples illustrate the examples of the claimed cosmetic compositions of the present invention but are not intended to be limiting thereof:

### Example 1

Basecoat

| Ingredient | Weight (%) |
|---|---|
| Group A: | |
| Silicone Gum¹ | 10.91 |
| Isododecane² | 50.00 |
| Group B: | |
| Organosiloxane resin³ | 19.09 |
| Red #6 Calcium Lake | 3.00 |
| Red #7 Barium Lake | 3.00 |
| Titanium Dioxide | 3.00 |
| Brown Iron Oxide | 1.00 |
| Bentone Gel⁴ | 10.00 |

¹1,000,000 cSt Dimethicone Gum available as 200 series fluid from Dow Corning.
²Permethyl 99A available from Permethyl Corporation.
³MQ Resin (0.7:1 ratio M:Q) available as TP3744 from General Electric (premix in isododecane). Amount in formulation is of resin only, not premix.
⁴VS-5 PC available from Elementis.

Combine Group A ingredients together in a beaker and mix with a propeller mixer until uniform. Add Group B ingredients to the Group A mixture and hand-mix to roughly incorporate the dry powders. Homogenize the entire formulation until all pigments are fully dispersed. Transfer the resulting fluid to individual packages.

Topcoat

| Ingredients | Weight (%) |
|---|---|
| Polybutene | 50.00 |
| Petrolatum | 39.00 |
| Mineral Oil | 8.00 |
| Candellila Wax | 3.00 |

Combine ingredients together in a beaker and mix with a propeller mixer until uniform. Heat ingredients to 60° C. to melt wax. Homogenize the entire formulation until all ingredients are fully dispersed. Cool and transfer the resulting product to individual packages.

### Example 2

Basecoat

| Ingredient | Weight (%) |
|---|---|
| Group A: | |
| Organosiloxane resin¹ | 19.20 |
| Isododecane² | 14.90 |
| Group B: | |
| Red #6 Calcium Lake | 1.49 |
| Red #7 Barium Lake | 2.10 |
| Titanium Dioxide | 2.33 |
| Brown Iron Oxide | 4.03 |
| Propylparaben | 0.15 |
| Group C: | |
| Silicone Gum³ | 10.80 |
| Isododecane² | 33.00 |
| Group D: | |
| Isododecane² | 10.00 |
| Trihydroxystearin | 2.00 |

¹MQ Resin (0.7:1 ratio M:Q) available as TP3744 from General Electric (premix in isododecane). Amount in formulation is of resin only, not premix.
²Permethyl 99A available from Permethyl Corp.
³Dimethicone Gum (25,000,000 cSt) available as SE 30 from General Electric.

Combine Group A ingredients together and mix with a propeller mixer until uniform. Add Group B ingredients to Group A mixture and homogenize until the pigments are completely dispersed. Premix Group C ingredients in a separate container using a propeller mixer until uniform, then combine with the mixture of Groups A and B ingredients. Premix Group D ingredients with heating to about 57-60° C. for about 3 minutes. Remove from the heat and homogenize for approximately five minutes or until a gel develops. Finally, add the Group D mixture to the rest of the batch and heat the entire mixture to 57-60° C. for about 7 to 10 minutes while mixing with a propeller mixer. Remove the batch from the heat and allow it to cool to room temperature while mixing with a propeller mixer. Transfer the resulting fluid to individual packages.

Topcoat

| Ingredients | Weight (%) |
|---|---|
| Polybutene | 40.00 |
| Petrolatum | 44.00 |
| Mineral Oil | 12.00 |
| Candellila Wax | 4.00 |

Combine ingredients together in a beaker and mix with a propeller mixer until uniform. Heat ingredients to 60° C. to melt wax. Homogenize the entire formulation until all ingredients are fully dispersed. Cool and transfer the resulting product to individual packages.

### Example 3

Basecoat

| Ingredients | Weight (%) |
|---|---|
| Group A: | |
| Silicone Gum¹ | 16.20 |
| Cyclopentasiloxane² | 70.00 |
| Group B: | |
| Organosiloxane resin³ | 1.80 |
| Red #6 Calcium Lake | 1.80 |
| Red #7 Barium Lake | 1.80 |
| Titanium Dioxide | 1.80 |
| Brown Iron Oxide | 0.60 |
| Bentone Gel⁴ | 6.00 |

¹25,000,000 cSt Dimethicone Gum available as SE 63 from General Electric.
²Gyclopentasiloxane 245 Fluid available from Dow Corning.
³MQ Resin (0.7:1 ratio M:Q) available as TP3744 from General Electric (premix in isododecane). Amount in formulation is of resin only, not premix.
⁴VS-5 PC available from Elementis.

Topcoat

| Ingredients | Weight (%) |
|---|---|
| Polybutene | 50.00 |
| Petrolatum | 39.00 |
| Mineral Oil | 8.00 |
| Candellila Wax | 3.00 |

Combine ingredients together in a beaker and mix with a propeller mixer until uniform. Heat ingredients to 60° C. to melt wax. Homogenize the entire formulation until all ingredients are fully dispersed. Cool and transfer the resulting product to individual packages.

### Example 4

| Ingredients | Weight (%) |
|---|---|
| Group A: | |
| Dimethicone Fluid¹ | 19.09 |
| Isododecane² | 20.00 |
| Group B: | |
| Organosiloxane resin³ | 33.42 |
| Red #6 Calcium Lake | 5.25 |
| Red #7 Barium Lake | 5.25 |
| Titanium Dioxide | 5.25 |
| Brown Iron Oxide | 1.74 |
| Bentone Gel⁴ | 10.00 |

¹1,000 cSt Dimethicone Fluid available from General Electric.
²Permethyl 99A available from Permethyl Corp.
³MQ Resin (0.7:1 ratio M:Q) available as TP3744 from General Electric (premix in isododecane). Amount in formulation is of resin only, not premix.
⁴VS5 PC available from Elementis

Combine Group A ingredients together in a beaker and mix with a propeller mixer until uniform. Add Group B ingredients to the Group A mixture and hand-mix to roughly incorporate the dry powders. Homogenize the entire formulation until all pigments are fully dispersed. Transfer the resulting fluid to individual packages.

Topcoat

| Ingredients | Weight (%) |
|---|---|
| Polybutene | 50.00 |
| Petrolatum | 39.00 |
| Mineral Oil | 8.00 |
| Candellila Wax | 3.00 |

Combine Group ingredients together in a beaker and mix with a propeller mixer until uniform. Heat ingredients to 60° C. to melt wax. Homogenize the entire formulation until all ingredients are fully dispersed. Cool and transfer the resulting product to individual packages.

### Method of Using the Invention

The method of using the present invention is straightforward. The user applies the composition of the present invention from a suitable liquid cosmetic applicator or a lip color bullet directly onto the skin. One such applicator used for liquid lip products is a liquid pen package disclosed in British Patent 21198037, issued May 9, 1990, assigned to Mitsubishi Pencil Co., Ltd. of Japan. An alternative package is one where a wand is dipped into a reservoir wherein the composition on the tip of the wand is applied to the skin surface. Such packages are disclosed in Japanese Utility Model 64 000822 Y2, to Shiseido.

Another cosmetic dispenser that is useful for the present invention is a unidirectional twist-up dispensing device with incremental dosing as disclosed in U.S. Pat. No. 5,851,079, issued on Dec. 22, 1998 to Richard L. Horstman et al. Such a twist-up dispensing device can include a hollow housing defining a chamber having an open dispensing end and a piston located within the chamber being limited to translational movement within the chamber. The piston preferably having a threaded rod extending there from that engages with a threaded aperture in an actuator such that advancement of the piston toward the dispensing end occurs when the actuator is rotated. Rotation of the actuator causes the product to be dispensed from the dispensing end. An applicator is preferably attached to the dispensing end of the housing in fluid communication with the chamber wherein the product is dispensed through the applicator. The applicator can comprise a ferrule and an application portion wherein the ferrule is attached to the dispensing end of the housing and the application portion has at least one orifice located therein. Several versions of applicators can be utilized including, for example, a fiber brush or an application surface having flocking thereon. Flocking is a mat of thin, short, plastic fibers substantially perpendicular to the application surface. The bristles of a fiber brush are preferably tapered and made of a plastic material. Alternatively, the user may use a more traditional applicator or implement known in the art, such as a dip in brush or doe foot applicator.

As stated above, the user applies the composition wherein the user allows the composition to dry before subjecting the composition to insult. Once the composition is dried, a complimentary product such as the plasticizing topcoat composition disclosed above may be applied over the dried product to provide the user with an aesthetically pleasing affect. Plasticizing topcoat compositions of the present invention may also utilize the same dispensing device.

The compositions of the present invention may be removed by applying petrolatum or a dimethicone-based cosmetic remover and by rubbing the area gently with a tissue to remove the cosmetic.

### Packaging System for Improving Film-Forming Basecoats

The present invention comprises a package system for improving film-forming basecoats including a two-step composition. One part of the package includes the basecoat of the present invention as described herein. The second part includes a topcoat plasticizing compositions that improves the film-forming basecoat whereby the resulting composition has a percent L value change of the basecoat film of less than about 50% and has a percent L value change of the transfer substrate greater than about 9% and no more than about 30%, as determined by the rub test.

The two compositions are packaged together, similarly, or in such a way that the consumer can identify the two compositions as those that are used together, in order to provide the consumer with a compatible, two-part cosmetic system with improved feel and appearance for long wearing lip color. The system results in a long wear performance that provides lubricity and shine.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-layer cosmetic composition comprising:
a. a transfer-resistant, film-forming basecoat composition formulated to be applied to keratinous tissue to form a basecoat film thereon; and
b. a topcoat composition comprising a plasticizing agent wherein the basecoat film, after application of said topcoat composition thereon, has a percent L value change of the basecoat film of less than about 50% and a percent L value change of a transfer substrate greater than about 9% to about 30%, as determined by the rub test.

2. The composition of claim 1, wherein said basecoat contains a polymers selected from silicone resins, silicone polymers, polysaccharides, polyurethanes, acrylates, polyesters, alkyds, fluoro polymers, and mixtures thereof.

3. The cosmetic composition of claim 1 wherein the plasticizing agent is selected from the group consisting of hydrocarbons, esters, silicones, modified silicones, glycols, fluid synthetic polymers, waxes, solid oils and mixtures thereof.

4. A method for improving the feel and aesthetics of transfer-resistant, film-forming basecoats wherein said method comprises the steps of:
d. applying a transfer-resistant, film-forming basecoat composition onto the surface of keratinous tissue to form a basecoat film;
e. allowing said basecoat to dry; and
f. applying over said basecoat a topcoat composition comprising a plasticizing agent wherein the basecoat film, after application of said topcoat composition thereon, has a percent L value change of the basecoat film of less than about 50% and a percent L value change of a transfer substrate greater than about 9% to about 30%, as determined by the rub test.

5. The method of claim 4 wherein said basecoat comprises polymers selected from silicone resins, silicone polymers, polysaccharides, polyurethanes, acrylates, polyesters, alkyds, fluoro polymers, and mixtures thereof.

6. The method of claim 4 wherein said basecoat comprises a fluid diorganosiloxane polymer and an organosiloxane resin and a volatile carrier.

7. The method of claim 4 wherein the composition is a lip product.

8. The method of claim 4 wherein the plasticizing agent is selected from the group consisting of hydrocarbons, esters, silicones, modified silicones, glycols, fluid synthetic polymers, waxes, solid oils and mixtures thereof.

9. The method of claim 8 wherein the hydrocarbons are selected from the group consisting of straight chain hydrocarbons, isoparaffins, mineral spirits, polybutene, hydrogenated polyisobutene, hydrogenated polydecene, polydecene, squalene, petrolatum and mixtures thereof.

10. The method of claim 8 wherein the silicones are selected from the group consisting of diorganosiloxanes, dimethiconols, cyclomethicones, alkyl silicones, phenyl trimethicone and mixtures thereof.

11. The method of claim 10 wherein the diorganosiloxane has a viscosity greater than about 1 cSt at 25° C.

12. A system for improving the feel and aesthetics of transfer-resistant, film-forming basecoats wherein said system comprises cosmetic compositions comprising:
g. a first composition, transfer-resistant, film-forming basecoat formulated to be applied to keratinous tissue to form a basecoat film thereon; and
h. a second, topcoat composition comprising a plasticizing agent wherein, the basecoat film, after application of said second, topcoat composition, has a percent L value change of the basecoat film of less than about 50% and a percent L value change of a transfer substrate greater than about 9% to about 30%, as determined by the rub test; and
i. instructions indicating that the composition of (a) should be used with the composition of (b).

13. The system of claim 12 wherein the composition of (a) is packaged separately from the composition of (b).

14. The system of claim 12 wherein the cosmetic compositions of (a) and (b) are packaged together in a unitary form.

15. The system of claim 12 wherein the compositions of (a) and (b) are lip products.

16. The system of claim 12 wherein said basecoat comprises polymers selected from silicone resins, silicone polymers, polysaccharides, polyurethanes, acrylates, polyesters, alkyds, fluoro polymers, and mixtures thereof.

17. The system of claim 12 wherein the plasticizing agent is selected from the group consisting of hydrocarbons, esters, silicones, modified silicones, glycols, fluid synthetic polymers, waxes, solid oils and mixtures thereof.

18. The system of claim 17 wherein the hydrocarbons are selected from the group consisting of straight chain hydrocarbons, isoparaffins, mineral spirits, polybutene, hydrogenated polyisobutene, hydrogenated polydecene, polydecene, squalene, petrolatum and mixtures thereof.

19. The system of claim 17 wherein the silicones are selected from the group consisting of diorganosiloxanes, dimethiconols, cyclomethicones, alkyl silicones, phenyl trimethicone and mixtures thereof.

20. The system of claim 19 wherein the diorganosiloxane has a viscosity greater than about 1 cSt at 25° C.
